(19) United States
(12) Patent Application Publication
(10) Pub. No.: US 2007/0253869 A1
(43) Pub. Date: Nov. 1, 2007

(54) CHEMICAL DELIVERY SYSTEM

(75) Inventors: Maxime Robert, L'Ancienne Lorette (CA); Timothy L. Giddens, Florissant, MO (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 11/734,848

(22) Filed: Apr. 13, 2007

Related U.S. Application Data
(60) Provisional application No. 60/796,026, filed on Apr. 28, 2006.

(51) Int. Cl. B01L 11/00

(52) U.S. Cl. 422/103; 422/100

(57) ABSTRACT

A device for dividing a fluid stream into a first stream and a second stream. The device is comprised of a housing defining an internal chamber. The housing has an inlet port, a first outlet port and a second outlet port. A first flow path through the housing connects the inlet port to the first outlet port. A second flow path through the housing connects the inlet port to the second outlet port. A first flow restriction is defined along the first path limiting flow along the first path.

### FIELD OF THE INVENTION

The present invention relates generally to medical washers/disinfectors for washing/disinfecting medical instruments and devices, and more particularly to a chemical delivery system for delivering chemicals to a washer/disinfector.

### BACKGROUND OF THE INVENTION

Medical washers/disinfectors are used to wash/disinfect medical, veterinary, and mortuary instruments and devices. Such washers/disinfectors typically include an injection pump to inject chemicals (detergents, lubricants and the like) into the medical washer. The injection pump is typically a peristaltic pump that is capable of operating at different injection rates for different types of chemicals. Most existing washers and their associated injection pumps are dimensioned to handle conventional chemicals and to inject such chemicals at a desired injection rate.

In recent years, ultra-concentrated chemicals have become available for use in medical washers. A problem with using such chemicals in existing medical washers is that the lowest injection rate for the injection pump is too high to inject these ultra-concentrated chemicals. In other words, even at the lowest pump speed, i.e., injection rate, too much of the ultra-concentrated chemicals would be injected into the medical washer.

The present invention overcomes this problem by providing a chemical delivery system for insertion into existing washers to enable ultra-concentrated chemicals to be used within a washer/disinfector with the existing injection pump.

### SUMMARY OF THE INVENTION

In accordance with a preferred embodiment of the present invention, there is provided a chemistry divider for dividing a stream of chemistry into a first stream and a second stream of chemistry. The divider is comprised of a housing defining an internal chamber and having an inlet port and first and second outlet ports. A flexible diaphragm is disposed within the chamber. The diaphragm separates the chamber into a first region and a second region. A first flow path through the housing connects the inlet port to the first outlet port. The first flow path communicates with the first region of the chamber. A second flow path through the housing connects the inlet port to the second outlet port. The second flow path communicates with the second region of the chamber. A valve element is connected to the diaphragm. The valve element is disposed in the second flow path and regulates flow therethrough based upon the position of the diaphragm.

In accordance with another aspect of the present invention, there is provided a flow divider for dividing a fluid stream into a first stream and a second stream. The divider is comprised of a housing defining an internal chamber. The housing has an inlet port and first and second outlet ports. A first flow path through the housing connects the inlet port to the first outlet port. A second flow path through the housing connects the inlet port to the second outlet port. A first flow control element is disposed along the first path for regulating flow along the path. A second flow control element is disposed along the second flow path for regulating flow along the path.

In accordance with yet another aspect of the present invention, there is provided a device for dividing a fluid stream into a first stream and a second stream. The device is comprised of a housing defining an internal chamber. The housing has an inlet port, a first outlet port and a second outlet port. A first flow path through the housing connects the inlet port to the first outlet port. A second flow path through the housing connects the inlet port to the second outlet port. A first flow restriction is defined along the first path limiting flow along the first path.

In accordance with still another aspect of the present invention, there is provided a chemical delivery system for delivering a chemical liquid to a medical washer, comprising a fluid feed line connectable at one end to a source of chemical liquid and connected at another end to a medical washer. A variable-rate injection pump is disposed in the fluid feed line. The injection pump has an inlet side and an outlet side. A flow-divider is disposed in the fluid feed line. The flow-divider has an inlet connected to the outlet-side of the injection pump, a first outlet connected to a portion of the fluid line connectable to the washer and a second outlet connectable to the fluid feed line at the inlet side of the injection pump. The flow-divider is comprised of a housing defining a first flow path connecting the inlet to the first outlet and a second flow path connecting the inlet to the second outlet. A flow restriction is disposed along the first flow path. The flow restriction limits fluid flow along the first path to a fraction of the output of the injection pump.

An advantage of the present invention is a chemical delivery system for dispensing chemicals into a washer/disinfector.

Another advantage of the present invention is a system as described above having a chemical injection device for reducing the flow of chemistry to a washer/disinfector.

Another advantage of the present invention is a chemical delivery system as described above that divides a chemical stream into two separate streams.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a schematic view of a medical washer/disinfector having a chemical delivery system therein;

FIG. 2 is an exploded view of a chemical delivery device used in the chemical delivery system shown in FIG. 1; and

FIG. 3 is an enlarged, cross-sectional view of the chemical delivery device shown in FIG. 2.

### DETAILED DESCRIPTION OF PREFERRED EMBODIMENT

Referring now to the drawings wherein the showings are for the purpose of illustrating a preferred embodiment of the invention only, and not for the purpose of limiting same, the drawing shows a medical washer/disinfector 10, a chemical delivery system 100 and a chemical delivery device 200 illustrating preferred embodiments of the present invention.

Broadly stated, washer/disinfector 10 includes a housing 22 that defines a chamber 24. Housing 22 is formed to include a sloped sump 26 that is disposed at the bottom of chamber 24. As will be described in greater detail below, sump 26 is provided to receive washing or rinsing fluids, designated “F” in the drawing. As referred to herein, the term “fluids F” refers to washing fluids, rinsing fluids, and any other fluid used in chamber 24. Heating elements 28 are provided in sump 26 to heat fluids “F.” A drain 32 is defined in the bottom of sump 26. A valve 34 is provided within drain 32 to control the flow of fluids “F” therethrough.

A circulation conduit 42 fluidly connects sump 26 to first and second branch conduits 44, 46 having spray heads 47 disposed therein. First branch conduit 44 is disposed in the upper portion of chamber 24 with spray heads 47 directed downward. Second branch conduit 46 is disposed in the lower portion of chamber 24 with spray heads 47 directed upward. In this respect, spray heads 47 direct fluids “F” toward the center of chamber 24, as illustrated in the drawing. A pump 52 is provided within circulation conduit 42 for pumping fluids “F” from sump 26 to spray heads 47. A motor 54, schematically illustrated in the drawing, drives pump 52.

Washer/disinfector 10 is dimensioned to contain one or more rack 48. Rack 48 is dimensioned to hold the instruments and equipment to be washed. Rack 48 is disposed between the upper and lower spray heads 47, as illustrated in the drawing.

A water supply line 62 fluidly communicates with washer/disinfector 10. Water supply line 62 has a first end 62a and a second end 62b. First end 62a is connectable to a water source (not shown) that is operable to provide clean water to washer/disinfector 10. Second end 62b of water supply line 62 is disposed within housing 22 above sump 26. A valve 64 is disposed within water supply line 62 and controls flow of water to sump 26.

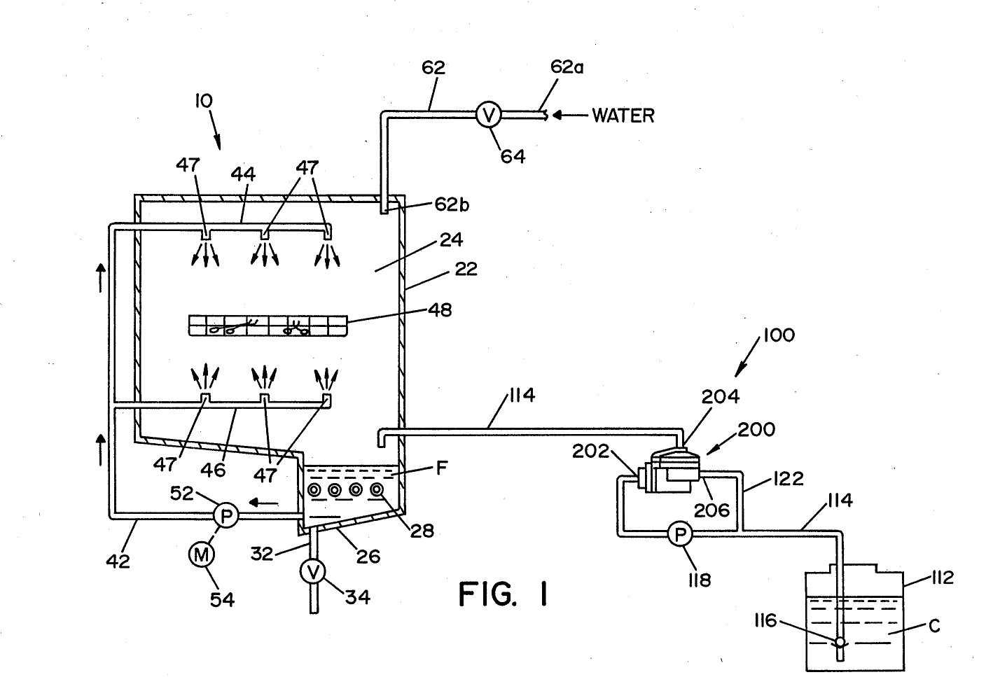

Chemical delivery system 100 includes a source 112 of a chemical composition, designated “C” in FIG. 1. Source 112 is connected to washer/disinfector 10 by a feed line 114. In a typical medical washer/disinfector, source 112 would be a replaceable container, such as a plastic bottle. Feed line 114 includes a directional check valve 116 to prevent back flow of fluid into source 112. An injection pump 118 is disposed in feed line 114 to inject the chemical composition “C” into washer/disinfector 10. Injection pump 118 is preferably a peristaltic pump that is capable of various injection rates, as is conventionally known. Other types of variable injection rate pumps are also contemplated.

The outlet side of pump 118 is connected to an inlet port 202 of chemical delivery device 200. Chemical delivery device 200 includes a first outlet port 204 connected to the section of feed line 114 that connects to washer/disinfector 10. Chemical delivery device 200 also includes a second outlet port 206 connected to a return line 122 that connects to feed line 114 on the inlet side of pump 118, as illustrated in FIG. 1.

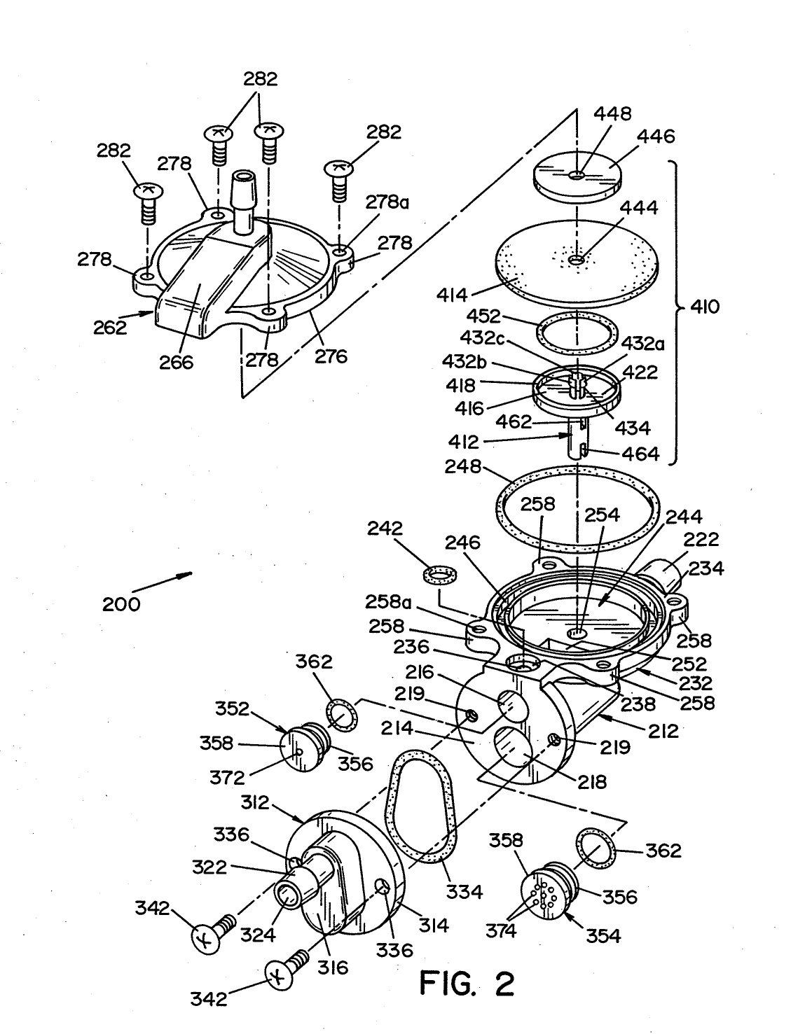

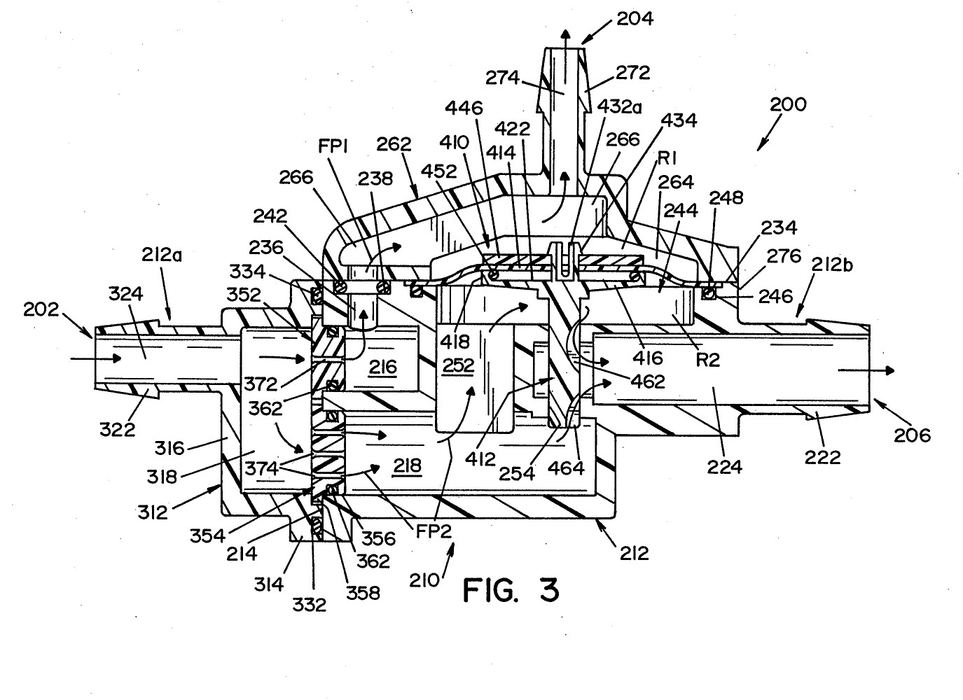

Referring now to FIGS. 2 and 3, chemical delivery device 200 is best seen. Chemical delivery device 200 is basically comprised of a housing 210 having a valve assembly 410 disposed therein. Housing 210 is comprised of a valve body 212, a cover plate 262, and an end plate 312. Valve body 212 includes a first end 212a and a second end 212b. First end 212a has a planar surface, having first and second cavities 216, 218 formed therein. In the embodiment shown, first and second cavities 216, 218 are generally cylindrical in shape, as best seen in FIG. 2. Cavities 216, 218 are side-by-side with second cavity 218 extending further into valve body 212, as best seen in FIG. 3. Threaded openings 219 are formed in planar surface 214 to extend into valve body 212, as best seen in FIG. 2. A fitting 222 is formed on second end 212b of valve body 212. In the embodiment shown, fitting 222 is a male nipple dimensioned to be attached to return line 122 that connects to feed line 114. An opening 224 extends through fitting 222 into valve body 212. Opening 224 in fitting 222 defines second outlet port 206 of chemical delivery device 200. An enlarged circular section 232 extends from one side of valve body 212. Circular section 232 has a planar surface 234, best seen in FIG. 2. A cylindrical bore 236 extends from planar surface 234 into valve body 212 to communicate with first cavity 216. Surface 234 is counter bored around bore 236 to define an annular shoulder 238 to receive an O-ring 242. A cylindrical recess 244 is formed in circular section 232 of valve body 212. A circular slot 246 is formed in planar surface 234 to surround recess 244. Slot 246 is dimensioned to receive an O-ring 248. A passage 252, best seen in FIG. 3, is formed within valve body 212 to connect second cavity 218 to cylindrical recess 244. A cylindrical, bored opening 254, best seen in FIG. 2, is centrally disposed within cylindrical recess 244. Bored opening 254 extends into valve body 212 and connects cylindrical recess 244 with opening 224 within fitting 222 and with cavity 218 within valve body 212. Circular section 232 of valve body 212 includes outwardly extending lugs 258, best seen in FIG. 2.

Cover plate 262 is generally circular in shape and defines an inner recess 264 dimensioned to correspond to, and mate with, cylindrical recess 244 in valve body 212. Cover plate 262 includes a channel 266 along one side thereof Channel 266 communicates with recess 264. A fitting 272 extends from the outer surface of cover plate 262. In the embodiment shown, fitting 272 is a male nipple dimensioned to be attached to the section of feed line 114 that connects to washer/disinfector 10. Fitting 272 has an opening 274 therethrough that communicates with channel 266 and recess 264 in cover plate 262. Fitting 272 defines first outlet port 204 of chemical delivery device 200. Cover plate 262 includes outwardly extending lugs 278 that are dimensioned to be in registry with lugs 258 on valve body 212. An opening 278a is formed in each lug 278. In this respect, cover plate 262 has a planar surface 276 dimensioned to mate with planar surface 234 of valve body 212. Cover plate 262 is attached to valve body 212 by conventional fasteners 282 extending through opening 278a of lugs 278 in cover plate 262 into threaded openings 258a formed in lugs 258 in valve body 212.

End cap 312 is dimensioned to be attached to planar surface 214 of valve body 212. End cap 312 includes a circular, generally flat plate portion 314 having an obround portion 316 extending to one side of plate portion 314. Obround portion 316 defines an internal obround or oval inlet chamber 318, best seen in FIG. 3. A fitting 322 extends to one side of obround portion 316. In the embodiment shown, fitting 322 is a male nipple dimensioned to be attached to feed line 114 from pump 118. An opening 324 through fitting 322 communicates with inlet chamber 318 and defines inlet port 202 of chemical delivery device 200. A groove 332, best seen in FIG. 3, is formed about the periphery of plate portion 314 to receive an oblong seal 334, best seen in FIG. 2. Holes 336 are formed through plate portion 314 of end cap 312. End cap 312 is dimensioned to be attached to planar surface 214 of valve body 212. Conventional fasteners 342 extending through holes 336 in plate portion 314 of end cap 312 extend into threaded openings 219 in valve body 212 to secure end cap 312 to valve body 212. As illustrated in FIG. 3, inlet chamber 318 is dimensioned to be in registry with first cavity 216 and second cavity 218 formed in valve body 212.

A first flow control element 352 is disposed within first cavity 216 of valve body 212, and a second flow control element 354 is disposed within second cavity 218 of valve body 212. In the embodiment shown, flow control elements 352, 354 are plugs or inserts dimensioned to be positioned within cavities 216, 218. In the embodiment shown, each element 352, 354 has a generally cylindrical body 356 and an outwardly extending flange 358 at one end thereof. An annular groove is formed in body 356 and is dimensioned to receive an O-ring 362. Flow control elements 352, 354 are dimensioned to be inserted into cylindrical cavities 216, 218, with flanges 358 of elements 352, 354 abutting planar surface 214 of valve body 212. O-rings 362 on elements 352, 354 form a seal between elements 352, 354 and valve body 212.

In the embodiment shown, flow control element 352 in cavity 216 in valve body 212 includes a single aperture 372 extending axially therethrough. Aperture 372 has a predetermined cross-sectional area. In the embodiment shown, aperture 372 is cylindrical in shape. Flow control element 354 in cavity 218 of valve body 212 has nine (9) spaced-apart, parallel apertures, each designated 374, extending axially therethrough. Each of the nine apertures 374 has a cross-sectional area equal to the cross-sectional area of aperture 372 in flow control element 352. As best seen in FIG. 3, when cover plate 262 is mounted to valve body 212, channel 266 within cover plate 262 communicates with bore 236 in valve body 212 connected to first cavity 216.

Valve assembly 410 is comprised of a valve stem 412 and a flexible diaphragm 414 that is attached to valve stem 412. Valve stem 412 is an elongated, cylindrical device having an outwardly extending flange 416 formed at one end thereof. Flange 416 includes an axially extending collar 418 that defines a shallow, circular recess 422. Beyond the flange, three (3) spaced-apart, axially-extending fingers 432a, 432b, 432c are formed. The ends of fingers 432a, 432b, 432c include outwardly extending barbs 434. Valve stem 412 is dimensioned to attach to diaphragm 414, best seen in FIG. 2. Diaphragm 414 is generally circular in shape and has a central opening 444 formed therein. Diaphragm 414 is preferably formed of a resilient, flexible material, such as neoprene rubber. Fingers 432a, 432b, 432c on the end of valve stem 412 are dimensioned to extend through opening 444 in diaphragm 414. A circular plate 446 having an outer diameter generally equal to the diameter of flange 416 is provided to attach to secure diaphragm 414 to valve stem 412. Plate 446 includes a centrally located opening 448 dimensioned to receive fingers 432a, 432b, 432c on valve stem 412. With diaphragm 414 mounted to fingers 432a, 432b, 432c of valve stem 412, plate 446 snaps onto fingers 432a, 432b, 432c and secures diaphragm 414 to valve stem 412, as illustrated in FIG. 3. An O-ring 452 is disposed within recess 422 of flange 416, as best seen in FIG. 3.

Diaphragm 414 is dimensioned to be captured between planar surface 276 of cover plate 262 and planar surface 234 of valve body 212.

As best seen in FIG. 3, a shallow recess is formed in circular section 232 of valve body 212 such that planar surface 276 of cover plate 262 rests on planar surface 234 of valve body 212 when the peripheral edge of diaphragm 414 is captured therebetween. Seal 248 that surrounds cylindrical recess 244 in valve body 212 helps form a seal between diaphragm 414, cover plate 262, and valve body 212. As illustrated in FIG. 3, valve stem 412 is dimensioned to be received within bored opening 254 in valve body 212 and to extend through opening 224 in fitting 222 into cavity 218 and valve body 212. Valve stem 412 is dimensioned to closely match the diameter of bored opening 254 in valve body 212 and to be slidable therein. In this respect, valve stem 412 is dimensioned to seal opening 224 in fitting 222 from recess 244 and cavity 218 in valve body 212. As best seen in FIG. 3, two spaced-apart slots 462, 464 are formed in valve stem 412. Slots 462, 464 are positioned on valve stem 412 to allow opening 224 in fitting 222 to be in fluid communication with recess 244 and cavity 218 in valve body 212, depending upon the position of valve stem 412 in bored opening 254, as shall be described in greater detail below.

As best seen in FIG. 3, when assembled, the cylindrical recess 244 in valve body 212 and recess 264 in cover plate 262 form a chamber within housing 210. Diaphragm 414 separates the chamber into a first region and a second region that are isolated from each other. In FIG. 3, the first chamber is shown above the diaphragm 414, and the second region is shown below the diaphragm 414.

The present invention shall now be further described with respect to the operation of chemical delivery device 200 and chemical delivery system 100. As indicated above, chemical delivery device 200 is disposed within feed line 114 such that inlet port 202 of chemical delivery device 200 receives the output from pump 118. Fluid flowing into inlet port 202 is directed to inlet chamber 318. From inlet chamber 318, the flow of fluid is separated into two separate streams.

A first stream or flow path “FP1” extends through aperture 372 in flow control element 352. Fluid flowing through flow control element 352 is directed into first cavity 216 within valve body 212. From cavity 216, the fluid flows through bore 236 into channel 266 formed in cover plate 262 and to first outlet port 204. Outlet port 204 is connected to the portion of feed line 114 that is connected to washer/disinfector 10. As can be seen in FIG. 3, fluid flowing along first flow path “FP1” communicates with one side of valve assembly 410, namely first region “R1” of the valve chamber.

A second stream or flow path “FP2” is defined by fluid flowing through flow control element 354 into cavity 218 in valve body 212. In this respect, fluid from inlet chamber 318 flows through the nine apertures 374 in flow control element 354 into cavity 218 in valve body 212. Through passage 252 connecting cavity 218 to the cylindrical recess 244 in valve body 212, fluid flowing along second flow path “FP2” communicates with the second side, i.e., a second region “R2,” of the valve chamber. Depending upon the position of valve stem 412 within opening 254 in valve body 212, fluid flows around valve stem 412 through the slots 462, 464 therein to outlet port 206 defined by fitting 222. As illustrated in FIG. 1, fluid flowing out of second outlet port 206 is directed through return line 122 back to feed line 114 at the inlet side of pump 118. Apertures 372, 374 in flow control elements 352, 354 basically define flow restrictions along first flow path FP1 and second flow path FP2, respectively.

Because flow control element 352 has only a single aperture 372, and flow control element 354 has nine like-dimensioned apertures 374, only one-tenth of the fluid injected into feed line 114 by pump 118 will flow along the first flow path through chemical delivery device 200 to washer/disinfector 10. Nine-tenths of the output of injection pump 118 is returned to the inlet side of injection pump 118. The present device thus meters the output of injection pump 118.

To ensure that only one-tenth of the output of injection pump 118 is injected to the washer/disinfector 10, it is necessary that the pressure within cavities 216, 218 be the same. A higher or lower pressure in one of cavities 216, 218 would produce a different flow through aperture 372 in flow control element 352, as compared to apertures 374 in flow control element 354. In this respect, valve assembly 410 is a pressure-equalizing device that balances the pressure in cavity 216 and cavity 218. In this respect, based on the operating characteristics of injection pump 118, the pressure on the first and second sides of diaphragm 414 will balance itself. Because apertures 374 through flow control element 354 insert have the same cross-sectional diameter as aperture 372 through flow control element 352, exactly one-tenth of the flow entering into inlet chamber 318 of chemical delivery device 200 will pass along the first flow path to washer/disinfector 10.

The present invention thus provides a device that can be easily inserted into existing washers to reduce the injection rate of chemical “C” to washer/disinfector 10, thereby allowing such washer/disinfector to utilize ultra-concentrated chemicals during a washing/disinfecting process. Installation requires that feed line 114 be split downstream from existing injection pump 118 and that the output end of injection pump 118 be attached to inlet port 202 of chemical delivery device 200. The other end of feed line 114, i.e., that portion of feed line 114 connected to washer/disinfector 10 is connected to first outlet port 204 of chemical delivery device 200. A T-fitting is then inserted into feed line 114 upstream of injection pump 118, and the leg of the T-fitting is connected to second outlet port 206 of chemical delivery device 200, so as to produce an arrangement as shown in FIG. 1. The present invention thus provides a simple method of retrofitting existing washers/disinfectors to allow for use of more recent developments in chemistry technology, namely ultra-concentrated chemical solutions. Still further, chemical delivery device 200 may be inserted in new washers/disinfectors to provide a wider choice of injection rates for an existing type of injection pump.

In accordance with another aspect of the present invention, flow control elements 352, 354 are removable from valve body 212 and may be replaced by like-shaped devices having different numbers of apertures extending therethrough. For example, in the embodiment shown, flow control element 352 has a single aperture 372 therethrough, and flow control element 354 has nine apertures 374 extending therethrough such that only ten percent (10%) of the fluid entering inlet chamber 3185 flows onto the washer/disinfector 10 along the first flow path. In other words, there are a total of ten apertures that allow fluid to exit inlet chamber 318, but only one aperture, i.e., aperture 372 in flow control element 352, allows flow to the washer/disinfector 10.

According to the present invention, various types of flow control elements, i.e., inserts, may be provided. For example, flow control element 352 may have two apertures therethrough, and flow control element 354 may have eight like apertures therethrough, wherein two apertures allow fluid flow to the washer/disinfector 10. As will be appreciated by those skilled in the art, other combinations of apertures can be utilized to form different flow rates through the chemical delivery device 200.

Still further, it is contemplated that each flow control element 352, 354 can be designed to have only a single aperture extending therethrough, wherein the cross-sectional area of one aperture is different than the other. For example, first flow control element 352 could be designed to have a single aperture 372 with a cross-sectional area “X,” and a flow control element 354 would be designed to have a single aperture 374 of cross-sectional area “2X.” Inserting such flow control elements 352, 354 into flow path FP1, FP2, respectively, of delivery device 200, would result in one-third of the fluid pumped into delivery device 200 flowing along first flow path FP1, and two-thirds of the fluid flowing along second flow path FP2.

It will also be appreciated that the cross-sectional shape of apertures 372, 374 in the aforementioned embodiments need not be circular. Other shapes may define the desired cross-sectional area(s).

Still further, it is contemplated that a single, removable flow control element can be used within housing 210 to regulate flow therethrough. For example, housing 210 may be formed to have an aperture of fixed cross-sectional shape therein between inlet chamber 318 and cavity 218. In other words, the aperture would be integrally formed as part of valve body 312. A single flow control element would be disposed in cavity 216. The cross-sectional area of the aperture in the flow control element in cavity 216 would be dimensioned relative to the fixed aperture in valve body 212 to produce the desired split in the first flow stream and the second flow stream. As in the foregoing embodiments, the flow control element in cavity 216 would be replaceable with other flow control elements having aperture(s) of other cross-sectional areas, so as to vary the amount of separation between the fluid flow along the first flow path FP1 and the second flow path FP2.

The foregoing description is a specific embodiment of the present invention. It should be appreciated that this embodiment is described for purposes of illustration only and that numerous alterations and modifications may be practiced by those skilled in the art without departing from the spirit and scope of the invention. It is intended that all such modifications and alterations be included insofar as they come within the scope of the invention as claimed or the equivalents thereof.

Having described the invention, the following is claimed:

1. A chemistry divider for dividing a stream of chemistry into a first and second stream of chemistry, said divider comprised of:
a housing defining an internal chamber and having an inlet port and first and second outlet ports;
a flexible diaphragm within said chamber, said diaphragm separating said chamber into a first region and a second region;
a first flow path through said housing connecting said inlet port to said first outlet port, said first flow path communicating with said first region of said chamber;
a second flow path through said housing connecting said inlet port to said second outlet port, said second flow path communicating with said second region of said chamber; and
a valve element connected to said diaphragm, said valve element disposed in said second flow path and regulating flow therethrough based upon the position of said diaphragm.

2. A chemistry divider as defined in claim 1, further comprising a first flow control element disposed along said first path for regulating flow along said path.

3. A chemistry divider as defined in claims 1 or 2, further comprising a second flow control element disposed along said second flow path for regulating flow along said path.

4. A chemistry divider as defined in claim 3, wherein said first and second flow control elements are inserts that are removable from said housing.

5. A chemistry divider as defined in claim 3, wherein said first flow control element is a removable insert having at least one aperture of a predetermined dimension formed therethrough.

6. A chemistry divider as defined in claim 5, wherein said second flow control element is a removable insert having a plurality of apertures of said predetermined dimensions formed therethrough.

7. A chemistry divider as defined in claim 3, wherein:
said first flow control element has an aperture therethrough connecting said inlet port to said first outlet port; and
said second flow control aperture having at least one aperture therethrough connecting said inlet port to said second outlet port.

8. A chemistry divider as defined in claim 7, wherein said aperture in said first flow control element has the same cross-sectional area as said at least one aperture in said second flow control element.

9. A chemistry divider as defined in claim 3, wherein said housing is comprised of a main body, a top cover and an end cap, said end cap having said inlet port formed therein, said top cover having said first outlet port formed therein, and said main body having said second outlet port formed therein.

10. A flow divider for dividing a fluid stream into a first stream and second stream, said divider comprised of:
a housing defining an internal chamber and having an inlet port and first and second outlet ports;
a first flow path through said housing connecting said inlet port to said first outlet port;
a second flow path through said housing connecting said inlet port to said second outlet port;
a first flow control element disposed along said first path for regulating flow along said path; and
a second flow control element disposed along said second flow path for regulating flow along said path.

11. A flow divider as defined in claim 10, wherein said first and second flow control elements are removable inserts in said housing.

12. A flow divider as defined in claim 10, wherein said first and second flow control elements each include at least one aperture extending therethrough.

13. A flow divider as defined in claim 12, wherein said apertures extending through said first and second flow control elements each having essentially the same cross-sectional area.

14. A flow divider as defined in claim 13, wherein the total cross-sectional area of the apertures through said first flow control element is different than the total cross-sectional area of the apertures through said second flow control element.

15. A flow divider as defined in claim 14, wherein the total cross-sectional area of the apertures through said first flow control element is less than the total cross-sectional area of the apertures through said second flow control element.

16. A flow divider as defined in claim 10, further comprising means for maintaining equal pressure in said housing downstream from said first and second flow control elements.

17. A flow divider as defined in claim 16, wherein said means for maintaining equal pressure is a valve assembly within said housing, said valve assembly comprised of a flexible diaphragm and a valve element connected to said diaphragm.

18. A flow divider as defined in claim 17, wherein said diaphragm separates said chamber into a first region and a second region, said first region communicating with said first flow path and said second region communicating with said second flow path.

19. A flow divider as defined in claim 18, wherein said valve element is movable with said diaphragm and is disposed in said second path, said valve element regulating flow along said second path based upon the position of said diaphragm.

20. A device for dividing a fluid stream into a first stream and a second stream, said device comprising:
a housing defining an internal chamber, said housing having an inlet port, a first outlet port and a second outlet port;
a first flow path through said housing connecting said inlet port to said first outlet port;
a second flow path through said housing connecting said inlet port to said second outlet port; and
a first flow restriction defined along said first path limiting flow along said first path.

21. A device as defined in claim 20, wherein said first flow restriction is a device disposed along said first flow path, said device having an aperture of predetermined cross-sectional area formed therethrough.

22. A device as defined in claim 21, wherein a second device is disposed along said second flow path, said second device having at least one aperture extending therethrough, said aperture defining a flow restriction along said second path.

23. A device as defined in claim 22, further comprising means for maintaining equal pressure in said housing along said first and second flow paths.

24. A device as defined in claim 23, wherein said means for maintaining equal pressure is a valve assembly.

25. A chemical delivery system for delivering a chemical liquid to a medical washer, comprising:
a fluid feed line connectable at one end to a source of chemical liquid and connected at another end to a medical washer;
a variable-rate injection pump disposed in said fluid feed line, said injection pump having an inlet side and an outlet side; and
a flow-divider disposed in said fluid feed line, said flow-divider having an inlet connected to said outlet-side of said injection pump, a first outlet connected to a portion of said fluid line connectable to said washer and a second outlet connectable to said fluid feed line at said inlet side of said injection pump, said flow-divider further comprising:
a housing defining a first flow path connecting said inlet to said first outlet and a second flow path connecting said inlet to said second outlet, and
a flow restriction disposed along said first flow path, said flow restriction limiting fluid flow along said first path to a fraction of the output of said injection pump.

26. A chemical delivery system as defined in claim 25, further comprising means for maintaining equal pressure in said housing along said first and second flow paths.

27. A chemical delivery system as defined in claim 26, wherein said means for maintaining equal pressure is a valve assembly within said housing, said valve assembly comprised of a flexible diaphragm and a valve element connected to said diaphragm.

28. A chemical delivery system as defined in claim 27, wherein said diaphragm separates said chamber into a first region and a second region, said first region communicating with said first flow path and said second region communicating with said second flow path.

29. A chemical delivery system as defined in claim 28, wherein said valve element is movable with said diaphragm and is disposed in said second path, said valve element regulating flow along said second path based upon the position of said diaphragm.

30. A chemical delivery system as defined in claim 25, wherein said flow restriction is defined by a flow control element disposed along said first flow path.

31. A chemical delivery system as defined in claim 30, wherein said flow restriction is a removable insert having an aperture therethrough, said aperture having a predetermined cross-sectional area.

32. A chemical delivery system as defined in claim 31, further comprising a second, removable flow restriction disposed along said second flow path, said flow control element having an aperture of a predetermined cross-sectional area extending therethrough.
